(19) United States
(12) Patent Application Publication
(10) Pub. No.: US 2006/0166951 A1
(43) Pub. Date: Jul. 27, 2006

(54) Compositions and methods for sterol isolation and purification

(75) Inventor: Alexandra J. Sanbom, Lincoln, IL (US)

Correspondence Address: KIRKPATRICK & LOCKHART NICHOLSON GRAHAM LLP;HENRY W. OLIVER BUILDING, 535 SMITHFIELD STREET, PITTSBURGH, PA, 15222, US

(73) Assignee: Archer-Daniels-Midland Company

(21) Appl. No.: 11/389,837

(22) Filed: Mar. 27, 2006

Related U.S. Application Data
(63) Continuation-in-part of application No. 10/447,212, filed on May 29, 2003.
(60) Provisional application No. 60/384,100, filed on May 31, 2002.

(51) Int. Cl. C07J 9/00; A61K 31/56

(52) U.S. Cl. 514/169; 552/545

(57) ABSTRACT

Compositions and methods for isolating and purifying sterols, stanols, policosanols, and other organic molecules of interest are disclosed. The compositions encompass purified sterols comprising, for example, policosanols. Methods comprise solvent extraction of sterols from any solute comprising sterol-containing material. Such sterol-containing material may be in the form of plant material such as crude tall oil, tall oil soap, or other suitable material.

### CROSS-REFERENCE TO RELATED APPLICATIONS

This application is a Continuation-In-Part of U.S. patent application Ser. No. 10/447,212 filed May 29, 2003, which claims the benefit of U.S. Provisional Application No. 60/384,100, filed May 31, 2002, the entire contents of both of which are hereby incorporated by reference in their entirety.

### FIELD OF THE INVENTION

This invention relates to the isolation and purification of organic molecules such as sterols, stanols, and policosanols from matter containing such molecules.

### SUMMARY

Compositions and methods for isolating and purifying sterols, stanols, policosanols, and other organic molecules of interest are disclosed. Methods comprise mixing a solvent with at least one sterol-containing material, precipitating at least some of the dissolved sterols, and recovering the precipitate of sterols. The precipitate may be further processed by dissolving the precipitate in a recrystallization solvent, re-precipitating at least some of the dissolved sterols, and recovering the precipitate of sterols. Solvents may comprise non-halogenated organic solvents such as heptane, hexane, isohexane, cyclohexane, pentane, methanol, ethanol, isopropyl alcohol, acetone, methyl ethyl ketone, diethyl ether, and ethyl acetate. In some embodiments, the solvent or combination of solvents may comprise water. Such methods are useful for isolating and purifying such molecules as sterols, stanols, and policosanols from products containing plant material. Thus, the methods are useful for isolating and purifying such molecules as sterols, stanols, and policosanols from wood pulping by-products, including, for example, crude tall oil and tall oil soap.

### BACKGROUND

Plants are a source of many chemical compounds which have health benefits. Some examples of such compounds are sterols (referred to as stanols where the sterol is saturated), policosanols, tocotrienols and tocopherols. For example, as early as the 1950s, researchers demonstrated that consumption of phytosterols reduced cholesterol in the blood (Pollak, Circulation 7:702-706 (1953)). Further, policosanols, such as octacosanol, a long chain fatty alcohol found in leaf cuticles, have been demonstrated to suppress lipid accumulation in rats fed a high-fat diet (Kato, et al., Br J Nutr 73:433 (1995)) and to inhibit platelet aggregation (Amruzazabala, et al., Thromb Res 69:321 (1993)). Tocotrienols and tocopherols, also found in plants, together comprise the Vitamin E family, which are vital for human health and development.

Plant-derived long chain fatty or waxy alcohols (generically referred to herein as policosanols) have been demonstrated as having biological activity including lipid effects as well as ergogenic effects with benefits in cardiovascular, cerebral, and muscular systems. Also, these compounds have activity as growth regulators for plants. 1-triacontanol, or myricyl alcohol, has been demonstrated as being a growth stimulant on a wide range of plants (U.S. Pat. No. 4,150,970). Recently, such compounds have been associated with inhibiting cholesterol biosynthesis and increasing LDL receptor-dependent processing (Menendez, et al., Biol. Res. 27:199 (1994); Brit. J. Nutrition, 77:923 (1997)) with such effects being demonstrated in patients with type II hypercholesterolemia and dyslipidemia associated with non-insulin dependent diabetes mellitus (Mirkin, et al., Int. J. Clin. Pharm. Res. 21:31-41 (2001)). Other applications that have been reported in the literature include platelet hyperaggregability, ischemia and thrombosis, prevention of drug-induced gastric ulcer and improvement of male sexual activity (see, for example, PCT Publication WO 94/07830).

Free primary alcohols are found in many plant waxes; e.g., in leaf bark and stem waxes of most plants. Natural plant waxes may be grouped into waxes of palms, grasses and sedges, broad-leaf trees, and narrow-leaf trees (Albin H. Warth in The Chemistry and Technology of Waxes, 1956, Reinhold Publishing Corporation, NY., pp. 76-341). The sugar cane, Saccharum officinarum, of the grass family, order Gramineae, has an appreciable deposit of wax on the surface of the stalks. This wax is of considerable economic value as it is rich in long chain fatty alcohols.

Numerous patents and patent applications describe methods for the isolation and purification of long chain fatty alcohols (see, for example, Japanese patent abstract, JP 60-119514; Japanese patent abstract, JP 62-87537; U.S. Pat. No. 5,856,316; and PCT Publication WO 94/07830). Waxes from plant sources have been fractionated into different classes such as hydrocarbons, second alcohols, esters, ketones, aldehydes, free alcohols and acids. Usually, long chain free alcohols in plants are present either as free alcohols or as esters of these alcohols with acids. Such esters usually contain an even number of carbon atoms in the range C₂₀-C₅₄. The long chain free alcohols usually found are straight chain primary alcohols and mainly of an even length (C₂₀-C₃₆). Sugar cane wax, for example, may contain up to 26% free and esterified long chain alcohols while Carnauba wax may contain up to 52.5% free and esterified long chain alcohols (Hamilton, et al, Plant Waxes in Topics in Lipid Chemistry, 3:199-269 (1972), F. D. Gunstone (ed.), John Wiley and Sons, Inc., NY).

It is well known in the literature that policosanols have anti-cholesterol effects. Gouni-Berthold et al. shows that doses of 10 to 20 mg per day of policosanol lowers total cholesterol (Gouni-Berthold, I. et al., Am. Heart J, 143:356-65 (2002)). U.S. Pat. No. 5,952,393 and U.S. Patent Publication No. 2001/0034338A1 disclose a purportedly synergistic anti-cholesterol effect of a composition comprising phytosterols and policosanols. U.S. Pat. No. 6,197,832 discloses the method of administering this composition of phytosterols and policosanols.

European Patent Application EP1 108 365 A2 discloses encapsulated policosanols for use in food applications.

U.S. Pat. No. 3,031,376 discloses the pharmaceutical use of certain long chain fatty alcohols in a method of increasing oxygen utilization.

European Patent Application EP1 108 363 A1 discloses processes for incorporation of long chain alcohols in edible oils.

European Patent Application EP1 108 364 A2 discloses a method for admixture of long chain alcohols in sterol compounds.

U.S. Pat. No. 6,277,403 discloses fat continuous emulsions containing, among other components, long chain alcohols having at least 20 carbon atoms in the alcohol chain.

U.S. Pat. No. 4,981,699 discloses a method for preparing an edible composition by extracting alcohols of 24-34 carbon atoms at subcritical or supercritical conditions and admixing peptides therewith.

U.S. Patent Application No. 2002/0016314A1 discloses compositions for reducing blood cholesterol and triglycerides comprising policosanol esters.

U.S. Pat. Nos. 5,865,316 and 5,663,156, and PCT Publication No. WO94/07830 disclose an extraction method of policosanols out of sugar cane wax and uses of those policosanol mixtures as a treatment for high cholesterol.

U.S. Pat. No. 6,328,998 discloses a method of treating high cholesterol with a pharmaceutically acceptable salt of L-carnitine and hexacosanol.

Sterols have also been shown to have an effect on blood cholesterol. Sterols are small organic molecules critical for many cellular processes, such as building and maintaining cell membrane integrity. Sterols are not only synthesized de novo by an organism but can also be derived from diet. One major source of dietary sterols for humans is phytosterols, which include both sterols and stanols found in plants. While elevated cholesterol is one of the well-established risk factors for coronary heart disease, it has been shown that a diet rich in phytosterols lowers this risk, as well as lowering the blood cholesterol level. As such, many food products can be enriched with phytosterols to improve human health and nutrition.

In fact, the United States Food and Drug Administration (“FDA”) has authorized the labeling of foods with health claims that state that plant sterols and plant stanols may reduce the risk of coronary heart disease by lowering blood cholesterol and low-density lipoprotein levels. Research has demonstrated that 1.3 grams per day or more of plant sterols or 3.4 grams per day or more of plant stanols in the diet show a significant cholesterol-lowering effect. A food should contain at least 0.65 grams of plant sterols per serving or at least 1.7 grams of plant stanols per serving to meet the FDA labeling requirements. Further, to meet European standards of sterol purity, such sterols must comprise less than 3% minor sterols. European Commission, Health and Consumer Protection; Scientific Committee on Food; Opinion Applications for Approval of a Variety of Plant Sterol-Enriched Foods (adopted by the Scientific Committee on Food, Mar. 5, 2003).

Commercial sources for phytosterols include vegetable oils such as soybean, sugar cane, and canola as well as by-products from the wood pulping process such as crude tall oil and tall oil soap, for example. In addition to sterols, policosanols can be isolated from such plant sources.

There are numerous other reports in the art for extraction of long chain alcohols from plant sources. For example, Staby and De Hertogh (Hortscience 74:411-412 (1972)) describe a process for separation of octacosanol in extracts from “Wedgwood” iris shots; Joshi and Singh report on the extraction of octacosanol from Gmelina arborea (Heartwoods) (Z. Naturforsch, 25:693-694 (1970)); Piatak and Reimann describe isolation of octacosanol from Euphorbia corollata (Phytochemistry, 9:2585-2586 (1970)). Other sources of free primary alcohols in oils and waxes have included germs, kernels and other components of nuts, seeds, fruits and cereals (Kawanishi, et al., JAOCS 68:869-872 (1991)).

Methods in the art have been described for the purification of long chain free alcohols. These include crystallization, chromatographic separation (Hamilton, et al., Plant Waxes in Topics in Lipid Chemistry, 3:199-269 (1972), F. D. Gunstone (ed.), John Wiley and Sons, Inc., NY), gel permeation chromatography (PCT Patent Application WO 99/48853), and multiple crystallizations in different solvents (U.S. Pat. No. 6,225,354; EP 0654262).

Various methods of extraction of sterols are also known in the art. In one extraction procedure disclosed in U.S. Pat. No. 6,057,462 to Robinson, et al., a solvent comprising heptane, methanol, and water is added to crude tall oil and then heated. The organic phase comprising the sterols is decanted and then cooled until the sterols precipitate. This precipitate is removed from the solvent using solid-liquid separation techniques that are well described in the art. However, procedures such as this require numerous processing steps and provide a product having a relatively low sterol yield; e.g., 80% of the sterols or less is recovered.

Thus, there exists a need in the art for more efficient methods of isolation and purification of sterols, policosanols, and other beneficial small molecules from plants. Procedures are especially needed that provide a relatively high yield, or ones provide fewer processing steps, or combinations thereof.

### DETAILED DESCRIPTION

It is to be understood that certain descriptions of the present invention have been simplified to illustrate only those elements and limitations that are relevant to a clear understanding of the present invention, while eliminating, for purposes of clarity, other elements. Those of ordinary skill in the art, upon considering the present description of the invention, will recognize that other elements and/or limitations may be desirable in order to implement the present invention. However, because such other elements and/or limitations may be readily ascertained by one of ordinary skill upon considering the present description of the invention, and are not necessary for a complete understanding of the present invention, a discussion of such elements and limitations is not provided herein. As such, it is to be understood that the description set forth herein is merely exemplary to the present invention and is not intended to limit the scope of the claims.

Other than in the examples herein, or unless otherwise expressly specified, all of the numerical ranges, amounts, values and percentages, such as those for amounts of materials, elemental contents, times and temperatures of reaction, ratios of amounts, and others, in the following portion of the specification and attached claims may be read as if prefaced by the word “about,” even though the term “about” may not expressly appear with the value, amount, or range. Accordingly, unless indicated to the contrary, the numerical parameters set forth in the following specification and claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains error necessarily resulting from the deviation found in its underlying respective testing measurements. Furthermore, when numerical ranges are set forth herein, these ranges are inclusive of the recited range end points (i.e., end points may be used). Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10.

Certain compositions within the present invention are generally described in the form of sterols that may be used in any dietary, nutritional, and medical product. It will be understood, however, that the present invention may be embodied in forms and applied to end uses that are not specifically and expressly described herein. For example, one skilled in the art will appreciate that embodiments of the present invention may be incorporated into any food or beverage.

All patents, publications, or other disclosure material referenced herein are incorporated by reference. Any patent, publication, or other disclosure material, in whole or in part, that is incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

The articles “a,” “an,” and “the” are used herein to refer to one or more than one (i.e., to at least one) of the grammatical objects of the article. By way of example, “a component” means one or more components, and thus, possibly, more than one component is contemplated and may be employed or used.

Compositions and methods for isolating and purifying sterols and other organic molecules of interest are disclosed herein. Compositions include sterols, stanols, policosanols, and combinations thereof. Methods comprise mixing a solvent with a solute such as sterol-containing material, and then cooling the mixture such that molecules of interest precipitate. Optionally, a solution may be heated prior to cooling and precipitation. The precipitate may be further purified through such procedures as recrystallization and chromatographic procedures. Sterols isolated using the methods of the invention may be used for any suitable purpose including, but not limited to, pharmaceuticals, foods, food additives, and beverages. Additionally, the sterols isolated using the methods of the invention may be further modified to produce chemical conjugates and derivatives, such as, for example, sterol and stanol esters. For example, in some embodiments, sterols derived from crude tall oil may be used to prepare fatty acid esters, for example, via hydrogenation to produce β-sitostanol, campestanol and stigmastanol and then via esterification with fatty acids derived from such vegetable oils to produce sterol fatty acid esters.

The methods of the invention are particularly advantageous in that the single or dual step processes can be performed using mild reaction conditions. Distillation is not required to achieve high levels of purity. Moreover, the procedures described herein can be performed at ambient temperature and pressure, not requiring extensive mechanical systems for purification. The methods described herein increase the efficiency of the extraction, requiring, for example, less solvent volume for extraction and washing. In addition, the methods provide a high yield and the purity of the recovered sterols, even over other more complex processes.

As used herein, the term “sterols” encompasses all sterols derived from any source and includes synthetic sterols, animal-derived sterols, and plant-derived sterols (hereinafter termed “phytosterols”), as well as the saturated forms of sterols thereof (i.e., stanols). Thus, the term “sterols” as used herein encompasses both sterols and stanols. Sterols are steroids with a hydroxyl group at C₃ (steroid alcohol) and most of the skeleton of cholestane (IUPAC Steroid Nomenclature, 1987). Additional carbon atoms may be present in the side chain, usually in the C₁₇ position. In nature, sterols are found as C₂₆-C₃₀ steroid alcohols. The cylcopentanoperhydrophenanthrene ring structure is common to all sterols, while the side chains may vary in structure. In nature, sterols may be found as conjugates (e.g., glycoconjugates, lipo-conjugates, etc.). Thus, the term “sterol,” as used herein, is further intended to encompass conjugated sterols, including, but not limited to, phytosteryl fatty-acid esters and phytostanyl fatty-acid esters.

The term “phytosterol” is intended to mean a sterol derived from plants and encompasses all plant sterols and the saturated forms of phytosterols thereof (i.e., phytostanols). Plant sterols fall into one of three categories: 4-desmethylsterols (lacking methyl groups); 4-monomethylsterols (one methyl group); and 4,4-dimethylsterols (two methyl groups) and include, but are not limited to, sitosterol (e.g., α and β sitosterol), campesterol, stigmasterol, taraxasterol, and brassicasterol. The term “phytostanol” is intended to mean a saturated phytosterol and encompasses, but is not limited to, sitostanol (e.g., α and β sitostanol), campestanol, stigmastanol, clionastanol, and brassicastanol. Phytosterols differ from animal sterols, in particular cholesterol, in that the side chain at position C₁₇ contains a double bond and sometimes an additional methyl, ethyl or ethylidene group, is present, in particular at position C₂₄. Sterols isolated by the methods of the invention may be quantified by any means known in the art, including, for example, the method described by Verleyen et al., JAOCS, 79:117 (2002). In certain embodiments, sterols and stanols are analyzed using a gas chromatograph-flame ionization detector (GC-FID) after first performing trimethylsilyl derivatization on the samples using BSTFA (N,O-bis(trimethylsilyl)trifluoroacetamide) with 1% TMCS (trimethylchlorosilane). In other embodiments, the methods of the invention comprise isolation of phytosterols from sterol-containing material. As used herein, the term “sterol-containing material” is intended to mean any material which can be a source of sterols, such as, for example, plant material. As used herein, the term “plant material” is intended to mean any material comprising plant matter. Such plant material may be processed plant products and by-products from such plant processing, such as, for example, vegetable processing streams. Thus, sterol-containing plant material of use in the methods of the invention may be: vegetable oils, such as soy oil, sunflower oil, canola oil, palm oil, and sugar cane oil; by-products of the Kraft wood pulping process, such as tall oil soap, crude tall oil; or any other suitable sterol-containing material.

The wood pulping process, also known as the Kraft process, involves heating wood chips in an aqueous solution of sodium hydroxide and sodium sulfide. Over time, the wood is delignified to produce a solution known as “black liquor.” In addition to lignin, black liquor contains cellulose pulp, rosin and fatty acid sodium soaps, organic products such as sterols and their fatty acid esters, diterpenes, policosanols, and stilbenes. Black liquor is further processed to remove the suspended cellulose pulp solids. The black liquor contains the fatty acids and rosin acids which have been saponified with strong alkali. Black liquor is further concentrated via evaporation. During evaporation the soap separates as curd, known variously as “tall oil soap,” “crude sulfate soap,” and “black liquor soap skimmings.” Tall oil soap typically has the following composition and properties: a) density, 7 lbs./gal. to 8.4 lbs./gal.; b) moisture, 30%-42%; c) total solids, 58% to 70%; d) lignin and pulp, 0.8% to 25%; e) tall oil, 48% to 60%; f) total alkali as NaOH, 8% to 9.2%; and g) free alkali as NaOH, 0.5% to 1.0%. To convert the tall oil soap into crude tall oil, sulfuric acid is mixed with the tall oil soap and the resulting oil is separated via settling. Approximately two pounds of tall oil soap yields one pound of crude tall oil. Crude tall oil may be further purified via distillation into tall oil rosin, tall oil pitch, and tall oil fatty acids.

In some embodiments, Kraft wood pulping process by-products are used as a source of phytosterols. For example, crude tall oil is composed of approximately 38% sterols and may comprise up to 20% to 30% solids. Phytosterols derived from crude tall oil generally contain a higher proportion of plant stanols (primarily β-sitostanol) than do vegetable oils. Tall oil additionally comprises tocopherols, tocotrienols, policosanols (long chain aliphatic alcohols), and other organic molecules. See Table 1. Sterol-containing material such as by-products from the Kraft wood pulping process, such as crude tall oil, may be derived from any woody plant variety, including pine (genus Pinus), spruce, oak, hemlock, fir, oak, or any other suitable sterol-containing woody plants.

The methods of the invention may comprise single or multiple solvent extraction steps. The procedure comprises mixing a sterol-containing material such as crude tall oil, vegetable oil, or any other sterol-containing material with a solvent. The solvent can be any suitable solvent that dissolves sterols or other molecules of interest. Such a solvent may be an organic solvent which includes but is not limited to an aliphatic solvent, an aromatic organic solvent, or a combination thereof. As used herein, the term “aliphatic” is intended to mean an organic substance which contains no benzene rings. Also, as used herein, the term “aromatic” is intended to mean an organic substance having one or more benzene rings. As used herein, the term “non-halogenated” is intended to mean lacking halogens such as chlorine, fluorine, bromine, and iodine. According to the methods of the invention, a chemical or chemical moiety thereof may be non-halogenated, and, thus, the term “non-halogenated” encompasses, for example, organic solvents lacking halogens. As used herein, the term “organic solvent” is intended to mean any solvent comprising carbon.

Solvents that find use in practicing the methods of the invention include aliphatic and cyclic aliphatic organic solvents, including the alkanes such as petroleum ether, hexane, isohexane, cyclohexane, heptane, and pentane, as well as the alkenes such as; aliphatic and cyclic aliphatic ketone organic solvents, including, for example, acetone, and methyl ethyl ketone; aliphatic and cyclic aliphatic ether organic solvents, including, for example, diethyl ether; aliphatic and cyclic aliphatic alcohols (including, for example, C₁-C₆ alcohols, such as isopropanol, propanol, butanol, methanol, and ethanol, as well as the oxidation products thereof); aldehyde, and carboxylic acid organic solvents; and aromatic hydrocarbons.

Organic solvents such as those described herein may be mixed together to further improve the efficiency of the extraction. The combination of such solvents may be performed in any order. Mixtures of particular use in the methods of the invention include, but are not limited to, binary solvents comprising a first and a second solvent. Such mixtures of solvents may be the same or different classes of solvents. Thus, the first solvent or the second solvent may be an organic solvent such as aliphatic and cyclic aliphatic hydrocarbon organic solvents (e.g., alkanes such as petroleum ether; hexane, isohexane, cyclohexane, heptane, and pentane); and alkenes containing, for example, 1-8 carbons; aliphatic and cyclic aliphatic ketone organic solvents (e.g., acetone, methyl and ethyl ketone); aliphatic and cyclic aliphatic ether organic solvents (e.g., diethyl ether); aliphatic and cyclic alcohols (e.g., C₁-C₆ alcohols such as isopropanol, propanol, butanol, methanol, and ethanol, as well as the oxidation products thereof), aldehyde, and carboxylic acid organic solvents; and aromatic organic solvents.

When a binary solvent system is employed, various volumetric ratios of the first solvent to the second solvent may be used in the practice of the methods of the invention. For example, the volumetric ratio of a first solvent to a second solvent may be 70:30; 80:20; 90:10; 91:9; 92:8; 93:7; 94:6; 95:5; 96:4; 97:3; 98:2; or 99:1. Such solvents can be combined into any suitable range. Thus, in one embodiment, suitable volumetric ranges of a first solvent to a second solvent are 90:10 to 99:1; 90:10 to 98:2; 95:5 to 99:1; and 95:5 to 98:2. The volumetric ratio of a second solvent in such binary solvents may range from 1% to 5% or more.

Those of skill in the art will understand that the ratios described herein may also be described as percentages, or vice versa. For example, a ratio of 90:10 (first solvent to second solvent), described supra, may also be described in the alternative as 90% of a first solvent and 10% of a second solvent. Both methods of description are mathematically identical. As such, one method of notation is not preferred over another and may in fact be used alone or in combination with an equivalent notation to describe the invention.

For example, in one embodiment, an aliphatic hydrocarbon organic solvent may be combined with an aliphatic ketone, such as a combination of heptane and acetone (i.e., heptane:acetone). In certain other embodiments, an aliphatic hydrocarbon organic solvent may be combined with an aliphatic alcohol such as heptane and methanol (i.e., heptane:methanol).

Solvent combinations are useful in the practice of the methods of the invention. For example, in certain embodiments, the solvent system may comprise heptane:acetone at a volumetric ratio of 98:2, or in some other embodiments, the solvent system may comprise heptane:acetone:water at a volumetric ratio of 95:4:1. In certain other embodiments, the solvent system may comprise heptane:methanol:water at a volumetric ratio of 95:4:1; 92:4:4; or 49:49:2. The volumetric ratio of water in binary and ternary solvents may range from 1% to 5% or more.

Moreover, in certain embodiments, the solvent or combination of solvents described herein may be mixed with water, which can be immiscible, partially miscible or entirely miscible in the solvent or combination thereof. Such combinations may be, for example, a binary solvent such as an aliphatic hydrocarbon solvent mixed with water, e.g., heptane:water; ethanol:water; and acetone:water. Additionally, the solvent combinations described herein may be combined with water to form a ternary solvent, such as, for example, heptane:acetone:water and heptane:methanol:water.

The methods of the invention comprise combining a solvent with a solute such as a sterol-containing material to form a mixture. As used herein, the term “solute” is intended to encompass any sterol-containing material combined with a solvent. The volumetric ratio of solvent to sterol-containing material, such as, for example, crude tall oil, can be any appropriate volumetric ratio, but may include volumetric ratios such as (solvent:solute) 50:1; 20:1; 19:1; 18:1; 17:1; 16:1; 15:1; 14:1; 13:1; 12:1; 11:1; 10:1; 9:1; 8:1; 7:1; 6:1; 5:1; 4:1; 3:1; 2:1; 1:1; 1:2; 1:3; 1:4; 1:5; 1:6; 1:7; 1:8; 1:9; 1:10; 1:11; 1:12; 1:13; 1:14; 1:15; 1:16; 1:17; 1:18; 1:19; 1:20; 1:50 or any volumetric ratio within the range there between. Ranges of solvent to solute volumetric ratios that are particularly useful include 10:1 to 20:1; 5:1 to 20:1; 1:1 to 20:1; 5:1 to 15:1; 1:1 to 15:1; 1:1 to 10:1; or any other suitable or effective range. The mixture of solvent and sterol-containing material may be continuously stirred or intermittently stirred to further increase the dissolution of the sterols. By the term “continuously stirred,” it is intended that the stirring provide a substantially uniform mixture. The term “substantially uniform mixture” is intended to mean that no significant phase separation occurs. A substantially uniform mixture as used herein includes, but is not limited, to both stable and unstable colloidal dispersions. Stirring may occur during heating of the mixture, during cooling of the mixture, during storage of the mixture, or of some combination thereof.

Temperatures suitable for partial or complete dissolution of the sterols can be any appropriate temperature, but may include ambient room temperature (rt), 22° C., 25° C., 30° C., 35° C., 40° C., 45° C., 50° C., 55° C., 60° C., 65° C., 70° C., 75° C., 80° C., 85° C., 90° C., 95° C., 100° C., or higher (depending on the boiling point of the solvent or solvent mixture). Moreover, those of skill in the art recognize that the temperature of a mixture is a function of the temperature of the components, i.e., solvent and solute (i.e., sterol-containing material). Thus, the appropriate temperature wherein complete or partial dissolution of sterols into the solvent occurs may be reached by adding components that have been combined to equilibrate to the appropriate temperature. Further, if the appropriate temperature is reached once the components are added together, the mixture may be heated or cooled to reach the appropriate temperature. For example, a suitable temperature wherein complete or partial dissolution of sterols occurs may be 25° C., 30° C., 35° C., 40° C., 45° C., 50° C., 55° C., 60° C., 65° C., 70° C., 75° C., 80° C., 85° C., 90° C., 95° C., 100° C., or higher. Suitable temperature ranges include, but are not limited to, 50° C. to 100° C.; 50° C. to 90° C.; 50° C. to 80° C.; 50° C. to 70° C.; 50° C. to 60° C.; 30° C. to 40° C.; 30° C. to 50° C.; 30° C. to 60° C.; 30° C. to 70° C.; 30° C. to 80° C.; 30° C. to 90° C.; and 30° C. to 100° C. The temperature may be adjusted as a function of pressure, especially where the temperature exceeds the boiling point of the solvent. Thus, useful pressures for heating the solvent and sterol-containing mixture include 1 atmosphere (atm), 2 atm, 3 atm, 4 atm, 5 atm, 6 atm, 7 atm, 8 atm, 9 atm, 10 atm, or more.

Where heating or cooling is used to raise or lower the temperature of a solvent, a sterol-containing material, a mixture, or some combination thereof, the rate of change of the temperature may be as a continuous gradient, a discontinuous (e.g., stepwise) gradient, or a combination thereof. In some embodiments, a mixture can be stirred during the heating process, the cooling process, or both. The heating process may be performed gradually over a suitable time. For example, in some embodiments, a mixture is heated over 1, 2, 3, 4, 5, 6, 7, 8, 9, 10, 11, 12, 14, 16, 18, 20, 22, and 24 hours or more. For example, in one embodiment, the heating process occurs until sterols become at least partially soluble in the solvent, for example, for 4 hours at a temperature between 60° C. to 70° C.

Following at least partial dissolution of the sterol component in the solvent system, the sterols in the mixture may be precipitated in any manner wherein the solubility of the sterols (the solute) in the solvent-solute mixture is reduced, such as by cooling. In certain embodiments, such as where a mixture is cooled to 0-5° C., co-precipitation of glycerides and tocopherols may occur. In certain embodiments, these additional precipitates may be considered impurities. However, in other embodiments, the co-precipitates may be desirable products in the precipitate. Thus, those of skill in the art will recognize that the cooling temperature may be adjusted to change the composition of the precipitate based on the desired composition. Such cooling temperatures include, but are not limited to, 35° C., 30° C., 27° C., 26° C., 25° C., 24° C., 23° C., 22° C., 21° C., 20° C., 19° C., 18° C., 17° C., 16° C., 15° C., 14° C., 13° C., 12° C., 11° C., 10° C., 9° C., 8° C., 7° C., 6° C., 5° C., 4° C., 3° C., 2° C., 1° C., and 0° C. Cooling temperature ranges of particular use in the practice of the methods of the invention include cooling from 30° C. to 0° C.; to room temperature or below, e.g., 30° C. to 20° C.; room temperature (i.e., 22° C.) to 5° C.; 22° C. to 10° C.; 22° C. to 15° C.; 22° C. to 20° C.; 15° C. to 5° C.; and 15° C. to 10° C.

The cooling rate may be a gradient, like the heating rate, which can be continuous, discontinuous (e.g., stepwise), or a combination thereof. The cooling may be performed gradually over a suitable time. For example, in some embodiments, the mixture is cooled over 1, 2, 3, 4, 5, 6, 7, 8, 9, 10, 11, 12, 14, 16, 18, 20, 22, and 24 hours or more. The mixture may be stirred during the cooling process. For example, in one embodiment, the cooling process is performed with continuous stirring over four hours. Once the mixture has cooled, it may remain at a target temperature. For example, in one embodiment, the mixture may be allowed to remain at ambient room temperature or lower (e.g., 22° C., 5° C., or 0° C.) once the cooling process is complete. In some embodiments, the cooling time is 30 min. In other embodiments, the cooling time is 1 hour. In yet other embodiments, the cooling time is 4 hours. In some other embodiments, the cooling time is 6 hours.

During precipitation, such as by cooling, sterols precipitate as crystals. The precipitate, which is a solid, may then be separated from the liquid by any suitable means, including filtration (e.g., Buchner funnel) and centriftigation. The percent recovery by weight of total sterols from the original sterol-containing material is termed “yield.” Using the methods of the invention, yields of sterols recovered from the source material may be greater than 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or more, by weight. Further, solid yield may be calculated as a function of mass and purity; i.e., yield=(weight of solid obtained multiplied by the percent by weight of sterols in solid) divided by (weight of starting sterol-containing material multiplied by the percentage weight of sterols in sterol-containing material).

Using the methods of the invention, a precipitate comprising sterols may further comprise other organic molecules of interest, including, but not limited, to stanols, policosanols, tocopherols, and tocotrienols. As used herein, the percentage of sterols by weight comprising the precipitate is termed “purity.” Using the methods of the invention, the purity of sterols recovered from the sterol-containing material may be greater than 50%, 55%, 60%, 65%, 70%, 75%, 80%, 85%, 90%, 91%, 92%, 93%, 94%, 95%, 96%, 97%, 98%, 99%, or more, by weight.

Where purity of sterols in the precipitate is less than 100%, the remaining mass may be comprised of other molecules such as policosanols, tocopherols, tocotrienols, and combinations thereof. When present in the compositions of the invention, policosanols may be any carbon length, but may comprise a carbon length of at least C₂₀, such as, for example, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, and C₃₆. When present in the compositions of the invention, tocopherols may comprise alpha, beta, gamma, and delta tocopherols. When present in the compositions of the invention, tocotrienols may comprise alpha, beta, gamma, and delta tocotrienols. When policosanols, tocopherols, or tocotrienols or other molecules of interest are present either alone or in combination with the composition of the invention, they may comprise 3%, 2.5%, 2%, 1.5%, 0.5%, 0.25%, 0.1%, or less of the compositions of the invention by weight. Such policosanols, tocopherols, tocotrienols, or other molecules of interest may be found in certain embodiments of the invention at concentration ranges such as 0.6% to 0.2%; 0.5% to 0.1%; 1.0% to 0.1%; 1.5% to 0.1%; 2.0% to 0.1%; 2.5% to 0.1%; and 3.0% to 0.1%. Tocotrienols, tocopherols, policosanols, or some combination thereof, which may be present in the compositions of the inventions, can confer additional advantages, such as, for example, additional health benefits when the compositions of the invention are used as an additive, pharmaceutical, or food supplement. Thus, in some embodiments of the invention, the methods of the invention yield a composition that can be used in the manufacture of products that can provide health benefits of both sterols and another chemical compound such as tocotrienols, tocopherols, policosanols, or some combination thereof. Thus, in some embodiments, the methods of the invention that may be used to provide novel compositions that provide synergistic health effects. For example, Gouni-Berthold discloses a purportedly synergistic anti-cholesterol effect of a composition comprising phytosterols and policosanols. See, for example, Gouni-Berthold, I. et al., Am. Heart J, 143:356-65 (2002); U.S. Pat. No. 5,952,393 and U.S. Patent Publication No. 2001/0034338A1).

The sterols recovered from the sterol-containing material using the methods of the invention may be used for any purpose or may be further purified. For example, the recovery product from a first precipitation step, as described herein, may be washed to remove excess solvent or adherents. Such washes may be performed using any suitable solvent, including, but not limited to, water, alcohols (e.g., methanol), aliphatic hydrocarbon organic solvents (e.g., heptane), and solvents as described elsewhere herein, using any suitable means. For example, in certain embodiments, the wash solvent is methanol or heptane:methanol:water (95:4:1). Such a wash step is optional and may be performed at any time once the sterols have been recovered. Further, residual solvent may be removed from the precipitate to create a desolventized product using any suitable means, such as evaporative drying, e.g., vacuum evaporation on a rotary evaporator at 25 mm Hg.

In certain embodiments, the precipitate recovered from an initial solvent extraction step may be further purified using additional steps. For example, in some embodiments, the precipitate recovered from an initial solvent extraction-precipitation step, as described herein, may be redissolved in a suitable solvent and then recrystallized. Such a step may be performed in any suitable solvent, including, but not limited to, the solvents and combinations of solvents disclosed herein for an initial solvent extraction-precipitation step. In certain other embodiments, the solvent used to re-precipitate the sterols is heptane or heptane:methanol:water (95:4:1). Further, such a recrystallization procedure may be performed at the processing parameters and conditions, such as at the temperatures and for the times described herein for an initial extraction-precipitation procedure, as described supra. Such a recrystallization step is optional and may be performed at any time with the precipitate recovered from an initial solvent extraction, as described herein, or in any method known in the art.

Without intending to limit the scope of the present invention, in certain embodiments, a suitable solvent is combined with crude tall oil at a suitable volumetric ratio, such as those ratios set forth herein, such as 10:1; 5:1; and 3:1, while being continuously mixed and heated from ambient room temperature to a temperature of 60° C. In such embodiments, a suitable solvent is heptane:methanol:water (e.g., at a volumetric ratio of 95:4:1). Further, in such embodiments, the mixture is then cooled to 20° C. over a sufficient time (e.g., 1 hour or more) to cause the sterols to precipitate. Advantageously, a precipitate of sterols in some of the embodiments of the invention is recovered at a yield of between 85% to 95% by weight and a purity of 95% to 100% by weight. In certain embodiments where the sterols are less than 100% pure, the only identifiable organic molecules are the policosanols, and as discussed supra, it has been shown that plant molecules such as policosanols also have health benefits.

In certain embodiments, an initial purification of a sterol-containing source material may be performed using chromatography. Such a chromatography step may be performed in addition to, or in lieu of, any other isolation process described herein. For example, in one embodiment, sterols from a sterol-containing source material (e.g., crude tall oil) are isolated using silica gel chromatography. Thus, in certain embodiments, the source material is placed on the silica gel column (C-GEL C-560™, Zeochem) and eluted using a heptane-acetone gradient. For example, in one embodiment, a heated column is loaded with approximately 400 g of C-GEL C-560™ in heptane. A feed of 20 g of crude tall oil (38% sterols) in 100 mL heptane is heated to 50° C. and fed to the heated column (45-50° C.) at a rate of 7 mL/min. In some embodiments, the diluent may contain as much as 96.63% sterols or higher.

This invention further relates to a method of extracting the policosanols from various sources. It is well known in the art that these policosanols can be extracted from beeswax, rice bran oil, and sugar cane mud; however, it is one object of this invention that policosanols can also be extracted from vegetable material/oilseeds. The method of extracting from such material is to collect the deodorizer distillate and other byproduct/processing streams created during the processing of oilseed for the production of the oils. Such novel sources are subjected to appropriate extraction/separation techniques for isolation and purification of policosanols.

In one embodiment, the feed sources are subjected to solid-liquid extractions where the natural alcohols mixture is selectively extracted with adequate organic solvents. Examples of suitable solvents include toluene, heptane, acetone, methanol, ethanol, chloroform, butanone, propanol, ethyl acetate, and others including their mixtures. The extraction may be carried out from periods ranging from 6 to 24 h. The product may be recrystallized using the above mentioned solvents and mixtures thereof and may be subjected to vacuum drying. A pressure of about 25 mm Hg may be used during the vacuum drying stage. The purity of the obtained policosanols mixtures may be at least 50%, or at least 60%, or at least 70%, or at least about 80%, or at least 90%, with alcohols ranging from 22 to 34 carbons. The melting point range of attained alcohol mixtures maybe 81.7 to 86.3° C.

In another embodiment, the feed material may be mixed with the solvent of choice and heated to a temperature between 45° C. to 70° C., or in another embodiment, between 50° C. to 65° C., or, in yet another embodiment, 60° C. to give a uniform solution and gradually cooled. The mixture may be stirred at room temperature, 5° C. or 0° C.

In a further embodiment, a second stage purification procedure may be performed to purify the recrystallized molecules of interest. The second stage purification can be performed using several different procedures, including solid-liquid extraction, Soxhlet extraction, normal phase chromatography, reverse phase chromatography, and complexation. The specific procedure in the complexation studies entails mixing of the solid extract with calcium bromide in methyl isobutyl ketone (MIBK) at constant heating (at a temperature between 45° C. to 70° C., or in another embodiment, between 50° C. to 65° C., or, in yet another embodiment, 60° C.) and stirring for about 1 hour. Following filtration, the solid may be washed and placed under vacuum at a temperature between 45° C. to 70° C., or in another embodiment, between 50° C. to 65° C., or, in yet another embodiment, 60° C. for 12 hours. The solid may then be stirred in water and filtered to give the product sample. The sample may then be washed with a solvent of choice. Washing of the solid extract with either diisopropyl ether or methyl ethyl ketone can provide a filtrate of enhanced policosanol content.

The proposed procedure for obtaining the higher molecular weight primary aliphatic alcohols has some advantages with regards to those previously reported. One of these advantages is related to its simplicity such that this process is more appropriate for large scale production. Still another advantage of this invention is related to the high degree of purification from a starting source of significantly lower percentage of policosanols relative to those of prior art. Other advantages are related with the degree of purity and the practicality of yield than those reported by the state of the art.

The compositions of the present invention are unique in that they are derived from novel source materials, and particularly from vegetable oil/oilseed processing streams and/or byproducts. Soy, corn, linseed, sunflower, rice, flax, wheat, and rape seed processing, for example, have been found by the present inventors to give rise to byproduct streams comprising policosanols containing various relative amounts of the long chain fatty alcohols. Policosanols and other molecules of interest can also be derived from other sources, including oils such as tall oil. Various deodorizer distillates are potential sources. Certain vitamin E processing streams have also been found to contain policosanols, as have filtrates obtained from sterol isolation of deodorizer distillates.

A plant source such as portions of cereals like soybean, linseed, sunflower and wheat may serve as a raw material for extraction. Alcohols may be extracted from the wax portions of these plants, which are recovered usually in their processing. For instance, the waxes from linseed and sunflower are usually extracted with linseed oil and sunflower oil as part of that process. The vegetable oils produced by extraction are usually winterized to improve clarity and appearance of the product. The process of winterization of the vegetable oil involves chilling of the oil to precipitate the waxes followed by filtration with diatomaceous earth to remove them. It has been discovered that such streams of filter aids and other filter media may serve as a source of long chain alcohols. These sources are presently land filled for lack of a better mechanism for disposal. By practicing the embodiments of the present invention those skilled in the art will be able to extract streams rich in long chain alcohols from such sources. Other sources of such compounds may be streams in wheat milling operations that are rich in the wheat germ fraction. Wheat germ is high in oil content and also contains long chain free alcohols. Such streams may serve as a method for providing the raw material for purification of long chain free alcohols.

The following are examples of methods and compositions of the invention. The examples are not meant to limit the scope of the invention, as defined by the claims.

### EXAMPLE 1

### Isolation of Sterols from Crude Tall Oil

Several experiments were conducted to isolate phytosterols from crude tall oil. Crude tall oil was composed of approximately 38% sterols as shown in Table 1 (data from two independent batches). The results showed that the purification processes of the invention produced sterols of 99% purity or greater. In one solvent system, a mixture of heptane:methanol:water (95:4:1) produced a final product purity of 99% with an overall yield of approximately 90%.

TABLE 1
Analysis of Crude Tall Oil.

| | | Test 1 | Test 2 |
|---|---|---|---|
| Total Sterols (%) | | 37.62 | 39.24 |
| | Brassicasterol | 0.19 | 0.07 |
| | Campesterol | 3.83 | 2.89 |
| | Stigmasterol | 0.77 | 0.21 |
| | Sitosterol | 32.83 | 36.07 |
| Total Stanols (%) | | 5.64 | 5.12 |
| | Sitostanol | 4.67 | 4.61 |
| | Campestanol | 0.97 | 0.51 |
| Total Policosanols (%) | | 8.19 | 3.96 |
| | C₂₀ Alcohol | 0.80 | 0.7 |
| | C₂₁ Alcohol | * | * |
| | C₂₃ Alcohol | 0.10 | 0.03 |
| | C₂₄ Alcohol | 2.46 | 1.1 |
| | C₂₅ Alcohol | 0.06 | 0.05 |
| | C₂₆ Alcohol | 1.22 | 0.63 |
| | C₂₇ Alcohol | 0.03 | 0.01 |
| | C₂₈ Alcohol | 0.29 | 0.09 |
| | C₂₉ Alcohol | 0.34 | 0.33 |
| | C₃₀ Alcohol | 1.32 | 0.22 |
| | C₃₁ Alcohol | 0.54 | 0.13 |
| | C₃₂ Alcohol | nd | 0.07 |
| | C₃₃ Alcohol | nd | nd |
| | C₃₄ Alcohol | nd | 0.06 |
| | C₃₅ Alcohol | nd | 0.13 |
| | C₃₆ Alcohol | 0.59 | 0.17 |
| Total Tocopherols (%) | | 1.6 | 1.53 |
| | Delta Tocopherol | 0.05 | 0.03 |
| | Beta Tocopherol | 0.18 | 0.11 |
| | Gamma Tocopherol | 0.19 | 0.14 |
| | Alpha Tocopherol | 0.19 | 1.26 |
| Total Tocotrienols (%) | | 0.09 | 3.41 |
| | Delta Tocotrienol | 0.09 | 3.11 |
| | Beta Tocotrienol | nd | 0.08 |
| | Gamma Tocotrienol | nd | 0.03 |
| | Alpha Tocotrienol | nd | 0.18 |

nd = not detected;
* = Not assayed (C₂₁ alcohol is an uncommon policosanol)

The extractions comprised regulating a number of variables including solvent, concentration, temperature, and time. Generally, crude tall oil was mixed with the solvent of choice, followed by heating to approximately 60° C. to produce a uniform solution and followed by gradual cooling. The mixture was then allowed to stir at room temperature, 5° C., or 0° C. The solid was isolated via Buchner filtration, washed, and dried under vacuum (25 mm Hg). Recrystallizations were performed similarly.

In a single solvent extraction, a mixture of heptane:methanol:water (95:4:1) and a gradient cooling system produced a product with greater than 98% purity with approximately 90% yield. See Table 2. In these experiments, sterols and stanols were analyzed using GC-FID after first performing trimethylsilyl derivatization of the samples using BSTFA with 1% TMCS. A 5% phenyl column (dimensions: 30 m×0.32 mm×0.10 μm) was used with helium as the carrier gas. Results were quantitated using an external standard calibration with an internal standard. The response factor from stigmasterol was used for all of the sterols. ISO (International Organization for Standardization) standard 12228 was used to identify the other sterol peaks.

In this example, crude tall oil was placed in the solvent mixture at a volumetric ratio of 5:1 (solvent:solute), heated to 55° C. with constant stirring, and then gradually cooled to room temperature over four hours. These results were confirmed using the two separate batches of crude tall oil as summarized in Table 2. A detailed description of the purity of the compositions of the isolated sterol by using the single-step procedure is shown in Table 3A. A distribution of residual policosanols and tocopherols from two test isolates is shown in Table 3B.

TABLE 2
Isolation of Sterols from Crude Tall Oil (CTO) Using a Single Extraction
with Heptane:Methanol:Water (95:4:1).

| | | SOLVENT:CTO | | STEROL | | | |
|---|---|---|---|---|---|---|---|
| | STARTING | VOLUMETRIC | TEMP | YIELD | POLICOSANOL | STEROL/STANOL | ERROR |
| EXPT # | BATCH | RATIO | °(C) | (%)¹ | PURITY (%) | PURITY (%) | (%) |
| 1 | Test 1 | 3:1 | rt | 83.98 | 0.99 | 95.31 | 0.76 |
| 2 | Test 1 | 3:1 | 60- | 90.10 | 2.19 | 98.08 | 1.42 |
| | | | gradient | | | | |
| 3 | Test 1 | 3:1 | 60- | 85.22 | 0.30 | 100.00 | 0.76 |
| | | | gradient | | | | |
| 4 | Test 2 | 3:1 | 60- | 93.72 | 0.47 | 97.26 | 1.51 |
| | | | gradient | | | | |
| 5 | Test 1 | 5:1 | rt | 87.11 | 0.32 | 99.16 | 1.42 |
| 6 | Test 1 | 5:1 | 60- | 89.33 | 0.29 | 100.00 | 0.76 |
| | | | gradient | | | | |
| 7 | Test 2 | 5:1 | 60- | 87.88 | 0.27 | 98.40 | 1.51 |
| | | | gradient | | | | |
| 8 | Test 2 | 5:1 | 60- | 91.44 | 0.46 | 100.19 | 0.15 |
| | | | gradient | | | | |
| 9 | Test 2 | 5:1 | 60- | 88.89 | 0.46 | 98.44 | 0.15 |
| | | | gradient | | | | |

¹Sterol yield = (amount of solid obtained × purity of sterols)/(amount of starting tall oil × purity of sterols in CTO).

TABLE 3A
Sterol Composition from a Single Heptane:Methanol:Water
(H:M:W; 95:4:1) Extraction as Shown in Table 2, supra.

| | Experiment # | | | |
|---|---|---|---|---|
| | 3 | 4 | 6 | 8 |
| Starting Batch | Test 1 (43% | Test 2 (44% | Test 1 (43% | Test 2 (44% |
| | sterol | sterol | sterol | sterol |
| | (including | (including | (including | (including |
| | stanol)) | stanol)) | stanol)) | stanol)) |
| Solvent:Solute | 3:1 | 3:1 | 5:1 | 5:1 |
| Volumetric | | | | |
| ratio | | | | |
| Wash | H:M:W | MeOH | H:M:W | MeOH |
| Phytosterol | 100.00 | 97.26 | 100.00 | 100.00 |
| Purity (%) | | | | |
| Overall Yield | 85.22 | 93.72 | 89.33 | 91.44 |
| (%) | | | | |
| Total Sterols | 88.06 | 85.01 | 88.54 | 88.92 |
| (%) | | | | |
| Total Stanols | 9.79 | 9.59 | 9.64 | 9.69 |
| (%) | | | | |
| Total Other | 2.25 | 1.66 | 2.21 | 1.58 |
| (%) | | | | |
| Brassicasterol | 0.06 | 0.04 | 0.05 | 0.05 |
| (%) | | | | |
| Campesterol | 7.32 | 5.71 | 7.28 | 5.82 |
| (%) | | | | |
| Campestanol | 0.99 | 0.71 | 0.95 | 0.71 |
| (%) | | | | |
| Stigmasterol | 1.24 | 0.38 | 1.34 | 0.55 |
| (%) | | | | |
| Sitosterol (%) | 79.46 | 78.88 | 79.88 | 82.50 |
| Sitostanol (%) | 8.80 | 8.88 | 8.69 | 8.98 |
| Total | 0.37 | 0.54 | 0.29 | 0.46 |
| Policosanols | | | | |
| (%) | | | | |
| % Error | 0.87 | 0.87 | 0.87 | 0.16 |

TABLE 3B
Concentrations of residual policosanols and tocopherols in two test
batches of isolated sterol.

| | Percent of Composition | Percent of Composition |
|---|---|---|
| Chemical Species | Isolate 1 | Isolate 2 |
| C20 Alcohol | nd | nd |
| C22 Alcohol | nd | nd |
| C23 Alcohol | nd | nd |
| C24 Alcohol | 0.08 | 0.08 |
| C25 Alcohol | nd | nd |
| C26 Alcohol | 0.13 | 0.11 |
| C27 Alcohol | nd | nd |
| C28 Alcohol | nd | nd |
| C29 Alcohol | 0.05 | 0.04 |
| C30 Alcohol | 0.14 | 0.17 |
| C31 Alcohol | 0.14 | 0.15 |
| C32 Alcohol | nd | nd |
| C33 Alcohol | nd | nd |
| C34 Alcohol | nd | nd |
| C35 Alcohol | nd | nd |
| C36 Alcohol | 0.02 | 0.02 |
| Delta Tocopherol | nd | nd |
| Beta Tocopherol | nd | nd |
| Gamma Tocopherol | 0.05 | 0.06 |
| Alpha Tocopherol | nd | nd |

nd = not detected

In further purification studies, it was shown that sterols of high purity (greater than 99%) could be isolated via several dual-stage processes as summarized in Table 4. Suitable results were obtained using a two-step purification process involving acetone extraction and crystallization, or heptane:methanol:water (H:M:W) extraction and crystallization followed by recrystallization with a heptane or heptane:methanol:water (H:M:W) or ethanol solvent system.

A first set of conditions using only an acetone:water solvent mixture also provided suitable results. Acetone was mixed with crude tall oil (5:1) with stirring, heated to 56° C., and then cooled, filtered, and rotary evaporated. The solid material (90% sterols) was then placed in a mixture of heptane:methanol:water (H:M:W; 95:4:1), heated, cooled, filtered, and washed to provide a final product of 99% sterol purity with 92% overall yield. The process was repeated with similar results.

A second set of conditions using only the heptane:methanol:water solvent mixture was also shown to provide suitable results. Initial extraction with this system (no gradient cooling) provided a material of 93% sterol purity with 91% yield. Recrystallization of the isolate with H:M:W gave a sterol mixture of greater than 98% purity and no loss in sterol recovery.

The processes outlined above were repeated using two different batches, and results were found to be reproducible. The final isolate is of greater than 98% sterol/stanol purity and was obtainable at approximately 90% overall yield. The only identified impurities in the isolated material were policosanols, which range from 0.25% to 1.24%. The analytical error percentage is reported as 0.15% to 1.66%.

TABLE 4
Summary of Acetone or Heptane:Methanol:Water (H:M:W; 95:4:1) Extraction
with a Second Extraction in H:M:W or Heptane for Sterol Isolation from Crude Tall Oil.

| | EXP. # | | | | | |
|---|---|---|---|---|---|---|
| | 10 | 11 | 12 | 13 | 14 | 15 |
| 1st Stage | Solid-Liquid | Solid-Liquid | Solid-Liquid | Solid-Liquid | Solid-Liquid | Solid-Liquid |
| Purification | Acetone (1:5) | Acetone (1:5) | H:M:W (1:5) | H:M:W (1:3) | H:M:W (1:3) | H:M:W (1:3) |
| 2nd Stage | Recrystallization | Recrystallization | Recrystallization | Recrystallization | Recrystallization | Recrystallization |
| Purification | H:M:W | H:M:W | H:M:W | heptane | heptane | heptane |
| Phytosterol | 99.65 | 98.72 | 99.48 | 100.00 | 99.99 | 100.88 |
| Purity (%) | | | | | | |
| Overall Yield | 90 | 92 | 91 | 79 | 82 | 81 |
| (%) | | | | | | |
| Total Sterols (%) | 83.90 | 83.34 | 87.31 | 90.81 | 88.64 | 91.57 |
| Total Stanols | 13.54 | 13.47 | 10.22 | 7.42 | 9.44 | 7.34 |
| (%) | | | | | | |
| Total Other (%) | 2.21 | 1.92 | 1.94 | 1.84 | 1.91 | 1.97 |
| Brassicasterol | nd | nd | nd | nd | nd | nd |
| (%) | | | | | | |
| Campesterol (%) | 7.58 | 7.56 | 6.97 | 7.58 | 7.58 | 7.68 |
| Campestanol (%) | 1.43 | 1.43 | 0.99 | 0.74 | 0.96 | 0.73 |
| Stigmasterol (%) | 0.89 | 0.84 | 1.23 | 1.33 | 1.34 | 1.38 |
| Sitosterol (%) | 75.43 | 74.93 | 79.10 | 82.51 | 79.72 | 82.51 |
| Sitostanol (%) | 12.11 | 12.04 | 9.24 | 6.61 | 8.48 | 6.61 |
| Total | 0.25 | 0.39 | 0.16 | 0.12 | 0.22 | 1.06 |
| Policosanols (%) | | | | | | |
| % Error | 0.87 | 0.87 | 0.87 | 0.87 | 0.87 | 0.87 |

### EXAMPLE 2

### Effect of Solvent System on the Isolation of Sterols from Crude Tall Oil

Purification studies showed that sterols of high purity (greater than 99%) can be isolated via several processes. See Table 5. The procedures involved solvent extraction and manipulation of a number of variables, including solvent, concentration, temperature, and time. The general procedure involved mixing crude tall oil with the solvent of choice, followed by to give a uniform solution and subsequent gradual cooling. The mixture was then allowed to stir either at room temperature, 5° C., or 0° C. The solid was isolated via Buchner filtration, washed, and dried under vacuum (25 mm Hg). Chromatographic purification was also performed by normal phase silica gel chromatography (C-GEL C560™, Zeochem) and elution with a heptane/acetone gradient.

Phytosterols of 100% purity with 67.8% yield were obtained using normal phase chromatography followed by ethanol (EtOH) recrystallization (Table 5, Experiment 16). In this experiment, the source material was placed on a silica gel column (C-GEL C-560™, Zeochem) and eluted using a heptane-acetone gradient. The heated column was loaded with approximately 400 g of C-GEL C-560™ in heptane. A feed of 20 g of crude tall oil (38% sterols) in 100 mL heptane was heated to 50° C. and fed to the heated column (45-50° C.) at a rate of 7 mL/min. The diluent contained as much as 96.63% sterols.

Solid-liquid extraction of crude tall oil with 90% ethanol followed by recrystallization with 95% ETOH provided a sterol product of 99% purity with 70.9% yield (Table 5, Experiment 17). High-purity sterols were also obtained with a solid-liquid extraction using EtOH and then recrystallization with a heptane:methanol:water solvent mixture (Table 5, Experiment 18). The yield with this process was 50.1%. Purification using only a heptane:methanol:water extraction without a subsequent recrystallization step provided sterols of 100% purity; however, the yield was 25% (Table 5, Experiment 19).

TABLE 5
Dual-Stage Isolation of Sterols from Crude Tall Oil Having 38% Sterols.

| | EXP # | | | |
|---|---|---|---|---|
| | 16 | 17 | 18 | 19 |
| 1^{st} Stage of | Chromatography C- | Solid-Liquid 90% | Solid-Liquid 90% | Solid-Liquid |
| Purification | Gel 560 | EtOH | EtOH | H:M:W |
| 2^{nd} Stage | Recrystallization | Recrystallization | Recrystallization | NONE |
| Purification | 95% EtOH | 95% EtOH | H:M:W | |
| Phytosterol | 101.06 | 99.17 | 101.07 | 100.71 |
| Purity (%) | | | | |
| Overall Yield | 67.80 | 70.90 | 50.10 | 25.05 |
| (%) | | | | |
| Total Sterols (%) | 88.96 | 85.65 | 90.93 | 92.50 |
| Total Stanols (%) | 12.10 | 13.52 | 10.14 | 8.21 |
| Brassicasterol | 0.04 | 0.04 | 0.04 | 0.07 |
| (%) | | | | |
| Campesterol (%) | 6.52 | 6.17 | 6.41 | 6.69 |
| Campestanol (%) | 1.01 | 1.12 | 0.82 | 0.69 |
| Stigmasterol (%) | 0.29 | 0.25 | 0.40 | 0.53 |
| Sitosterol (%) | 82.11 | 79.19 | 84.08 | 85.20 |
| Sitostanol (%) | 11.08 | 12.40 | 9.32 | 7.51 |

### EXAMPLE 3

### Effect of Solvent System and Additional Variables on the Isolation of Sterols from Crude Tall Oil

Several additional experiments were conducted to isolate phytosterols from crude tall oil. See Table 6. The procedures involved solvent extraction and manipulation of a number of variables, including solvent, concentration, temperature, and time. The procedure involved mixing of crude tall oil with the solvent of choice, followed by heating to give a uniform solution and subsequent gradual cooling. The mixture was then allowed to stir either at room temperature, 5° C., or 0° C. The solid was isolated via Buchner filtration, washed, and dried under vacuum (25 mm Hg).

TABLE 6
Isolation of Sterols from Crude Tall Oil (38% Sterols).

| | | | | | Sterol | |
|---|---|---|---|---|---|---|
| | | Solvent:CTO | | Time | Yield | Purity |
| Exp. # | Solvent | Ratio | Temp (C.) | (h) | (%)¹ | (%) |
| 20 | none | | | | | 38.1 |
| 21 | none | | | | | 40.0 |
| 22 | heptane | 5:1 | rt* | 3 | 5.01 | 92.4 |
| 23 | heptane | 10:1 | rt* | 3 | 10.0 | 74.1 |
| 24 | heptane:acetone | 3:1 | rt* | 3 | 9.01 | 91.4 |
| | (98:2) | | | | | |
| 25 | heptane:acetone | 5:1 | rt* | 18 | 11.32 | 87.6 |
| | (98:2) | | | | | |
| 26 | heptane:acetone | 5:1 | rt* | 18 | 11.32 | 87.7 |
| | (98:2) | | | | | |
| 27 | heptane:acetone | 10:1 | rt* | 18 | 11.05 | 83.8 |
| | (98:2) | | | | | |
| 28 | heptane:acetone:water | 5:1 | rt* | 18 | 0.00 | 0.0 |
| | (95:4:1) | | | | | |
| 29 | heptane:MeOH | 20:1 | 0 | 18 | 32.63 | 94.9 |
| | (95:5) | | | | | |
| 30 | heptane:water | 10:1 | rt* | 18 | 80.26 | 93.8 |
| | (94:6) | | | | | |
| 31 | heptane:MeOH:water | 5:1 | rt* | 48 | 0.00 | 0.0 |
| | (95:4:1) | | | | | |
| 32 | heptane:MeOH:water | 5:1 | 5 | 24 | 33.14 | 96.1 |
| | (95:4:1) | | | | | |
| 33 | heptane:MeOH:water | 10:1 | rt* | 48 | 40.26 | 103.0 |
| | (95:4:1) | | | | | |
| 34 | heptane:MeOH:water | 10:1 | rt* | 48 | 39.47 | 94.0 |
| | (95:4:1) | | | | | |
| 35 | heptane:MeOH:water | 10:1 | 5 | 3 | 9.21 | 91.8 |
| | (95:4:1) | | | | | |
| 19 | heptane:MeOH:water | 10:1 | rt* | 3 | 25.05 | 100.7 |
| | (95:4:1) | | | | | |
| 36 | heptane:MeOH:water | 4:1 | rt* | 18 | 75.96 | 96.0 |
| | (92:4:4) | | | | | |
| 37 | heptane:MeOH:water | 8:1 | rt* | 18 | 0.00 | 0.0 |
| | (49:49:2) | | | | | |
| 38 | EtOH:water | 3:1 | rt* | 22 | 100 | 64.3 |
| | (90:10) | | | | | |
| 39 | EtOH:water | 5:1 | rt* | 22 | 100 | 66.7 |
| | (90:10) | | | | | |
| 40 | EtOH:water | 10:1 | rt* | 21 | 81.58 | 76.5 |
| | (90:10) | | | | | |
| 41 | EtOH:water | 3:1 | rt* | 20 | 0.00 | 0.0 |
| | (70:30) | | | | | |
| 42 | EtOH:water | 5:1 | rt* | 20 | 0.00 | 0.0 |
| | (70:30) | | | | | |
| 43 | ethanol (95:5) | 3:1 | 60 | 6 | 100 | 97.1 |
| | | | gradient | | | |
| 44 | ethanol (95:5) | 3:1 | rt* | 24 | 54.68 | NA² |
| 45 | ethanol (95:5) | 5:1 | 60 | 6 | 92.16 | 97.1 |
| | | | gradient | | | |
| 46 | ethanol (95:5) | 5:1 | rt* | 24 | 50.68 | NA |
| 47 | ethanol (95:5) | 5:1 | rt* | 24 | 53.10 | NA |
| 48 | ethanol (95:5) | 10:1 | rt* | 18 | 48.16 | 93.5 |
| 49 | ethanol (95:5) | 20:1 | rt* | 24 | 17.37 | 97.0 |
| 50 | ethanol (95:5) | 15:1 | rt* | 18 | 19.74 | NA |
| 51 | isopropanol | 5:1 | rt* | 21 | 8.68 | 92.7 |
| 52 | isopropanol | 10:1 | rt* | 21 | 0.00 | NA |
| 53 | isopropanol | 10:1 | 5 | 21 | 44.74 | 88.4 |
| 54 | isopropanol | 5:1 | rt* | 20 | 9.74 | 92.7 |
| 55 | methanol | 3:1 | rt* | 20 | 100 | 59.0 |
| 56 | methanol | 5:1 | rt* | 3 | 100 | 78.7 |
| 57 | methanol | 5:1 | rt* | 3 | 100 | 72.5 |
| 58 | methanol | 5:1 | rt* | 20 | 100 | 64.2 |
| 59 | methanol | 10:1 | rt* | 48 | 100 | 75.0 |
| 60 | methanol | 20:1 | rt* | 12 | 100 | NA |
| 61 | acetone:water | 20:1 | rt* | 20 | 59.21 | 96.1 |
| | (95:5) | | | | | |
| 62 | acetone | 5:1 | rt* | 24 | 6.21 | NA |
| 63 | acetone | 10:1 | rt* | 24 | 0.00 | 0.0 |
| 64 | acetone | 10:1 | 5 | 48 | 43.95 | NA |
| 65 | acetone | 10:1 | rt* | 48 | 71.05 | 93.0 |
| 66 | acetone | 20:1 | rt* | 24 | 40.26 | 88.4 |

¹sterol yield = (weight of solid obtained multiplied by the percent by weight of sterols in solid) divided by (weight of starting sterol-containing material multiplied by the percentage weight of sterols in sterol-containing material)
²NA = not available.
*room temperature

Those of skill in the art understand that the best procedure to use for extraction of sterols from crude tall oil will depend on the desired product. For example, if a skilled artisan desires to use a one-step step extraction to achieve high purity, then the solvent system and procedures shown in Experiments 30, 32, 33, 40, 43, 45, 56, and 65 would be suitable, supra. However, if one of skill in the art desires a high yield in the first step and is willing to perform a second step for further purification then the solvent system and procedures using methanol or EtOH/water (90:10) would be suitable (e.g., Experiments 10, 11, 12, 13, 14, 15, 16, 17, and 18, supra).

### EXAMPLE 4

Various wax streams were obtained from the refining of vegetable oils from the Oil Processing Division of Archer-Daniels-Midland (ADM) Company, Decatur, III. These streams were obtained from the filtering step of the vegetable oil refinery where the oils are typically winterized or the deodorization step for purification of the vegetable oils. These processes are well known in the art. The streams analyzed were: Sun/Rape Deodorizer Distillate; Sunflower Deodorizer Distillate; Rape Distillate; Soy Deodorizer Distillate; Rape Seed Deodorizer Distillate; Canola Deodorizer Distillate; and Rice Bran Deodorizer Distillate. Each stream was found to contain at least 4 of the C₂₄-C₃₀ long chain fatty alcohols. See Table 7.

A portion of the material obtained from various sources was transesterfied using Ethanol Potassium Hydroxide to break the ester linkages between long chain alcohols and fatty acids. The streams were: Corn Dewax Cake; Rice Fatty Acid Distillate; Cotton Seed Deodorizer Distillate; Soy Hulls; Sunflower Hulls; Linseed Diatomaceous Earth; Sunflower Oil Dewaxing Cake C; and Sunflower Oil Dewaxing Cake. The composition of the different streams after esterification varied, as expected, but demonstrated the presence of C₂₀-C₃₀ alcohols in the ester form, suggesting that saponification could be used as an extraction process. See Table 8.

It will be apparent to those skilled in the art that these raw materials may be used as sources for production of a mixture of long chain alcohols. These long chain alcohols may be in free or esterified form and may occur with other phytochemicals such as sterols and tocopherols.

Policosanols, Sterols and Toconherols

TABLE 7

| | | | Rice | | | | | |
|---|---|---|---|---|---|---|---|---|
| | | Refined | Wax | | | | | |
| | Carnauba | Beeswax | from | Crude | Crude | Crude | Crude | Crude |
| | Wax from | from West | Punjab | Candelilla | Beeswax | Beeswax | Beeswax | Beeswax |
| Sample Id | Brazil | Africa | India | Wax | Africa | Brazil | China | Australia |
| Total | 0.03 | 2.14 | 0.13 | 3.134 | 1.85 | 0.787 | 0.869 | 0.826 |
| Tocopherols | | | | | | | | |
| Total Sterols | 0.04 | nd | 0.58 | 0.28 | nd | nd | nd | 0.082 |
| Total Stanols | nd | 0.36 | nd | 0.934 | 0.031 | 0.08 | 0.017 | 0.025 |
| Total | 10.66 | 1.18 | 1.06 | 4.106 | 0.244 | 0.34 | 0.261 | 0.4 |
| Policosanols | | | | | | | | |
| Delta | 0.03 | 2.10 | 0.13 | nd | nd | nd | nd | nd |
| Tocopherol | | | | | | | | |
| Beta | nd | nd | nd | nd | nd | nd | nd | nd |
| Tocopherol | | | | | | | | |
| Gamma | nd | nd | nd | nd | nd | nd | nd | nd |
| Tocopherol | | | | | | | | |
| Alpha | nd | 0.05 | nd | nd | 0.225 | 0.058 | 0.042 | 0.053 |
| Tocopherol | | | | | | | | |
| Brassicasterol | nd | nd | nd | nd | nd | nd | nd | nd |
| Campesterol | nd | nd | 0.10 | 0.041 | 0.18 | 0.044 | 0.039 | 0.045 |
| Campestanol | nd | nd | nd | 0.026 | 0.031 | 0.032 | 0.031 | 0.022 |
| Stigmasterol | 0.04 | nd | 0.22 | 0.208 | 0.237 | 0.067 | 0.073 | 0.082 |
| Sitosterol | nd | nd | 0.26 | 0.139 | 0.03 | 0.034 | 0.037 | 0.036 |
| Sitostanol | nd | 0.36 | nd | 1.32 | 0.59 | 0.262 | 0.283 | 0.311 |
| C20 Alcohol | nd | nd | nd | 0.313 | 0.02 | 0.034 | 0.027 | 0.089 |
| C22 Alcohol | nd | 0.02 | nd | 0.824 | 0.458 | 0.225 | 0.292 | 0.166 |
| C23 Alcohol | nd | nd | nd | nd | nd | nd | nd | nd |
| C24 Alcohol | 0.03 | 0.12 | 0.02 | 0.212 | 0.054 | 0.031 | 0.045 | 0.024 |
| C25 Alcohol | nd | nd | nd | nd | nd | nd | nd | nd |
| C26 Alcohol | 0.04 | 0.09 | 0.05 | 0.051 | 0.011 | nd | nd | nd |
| C27 Alcohol | nd | 0.03 | nd | nd | nd | nd | nd | nd |
| C28 Alcohol | 0.49 | 0.14 | 0.08 | nd | nd | nd | nd | nd |
| C29 Alcohol | 0.08 | 0.04 | nd | 0.267 | nd | nd | nd | nd |
| C30 Alcohol | 1.42 | 0.36 | 0.31 | 0.013 | nd | nd | nd | 0.082 |
| C31 Alcohol | 0.07 | 0.03 | nd | 0.061 | nd | nd | 0.008 | nd |
| C32 Alcohol | 6.23 | 0.31 | 0.33 | 0.751 | 0.02 | 0.08 | 0.009 | 0.025 |
| C33 Alcohol | 0.06 | nd | nd | 0.289 | nd | nd | nd | 0.03 |
| C34 Alcohol | 2.20 | 0.04 | 0.21 | 0.123 | nd | nd | nd | nd |
| C35 Alcohol | nd | nd | nd | nd | nd | nd | nd | nd |
| C36 Alcohol | 0.03 | nd | 0.06 | 3.817 | 0.244 | 0.34 | 0.261 | 0.37 |

Results are in g/100 g

TABLE 8
TRANSESTERIFIED: Results are % w/w

| | Sample Id | | | | |
|---|---|---|---|---|---|
| | C24 Alcohol | C26 Alcohol | C27 Alcohol | C28 Alcohol | C30 Alcohol |
| Rice Bran DOD 01 | 0.02 | 0.15 | 0.04 | 0.01 | 0.02 |
| Rice Bran DOD 02 | 0.02 | 0.13 | 0.03 | 0.01 | 0.02 |
| Rice Bran DOD 03 | 0.16 | 0.33 | 0.02 | 0.16 | 0.23 |
| Rice Bran DOD 04 | 0.17 | 0.34 | 0.02 | 0.15 | 0.23 |
| Corn Fiber | nd | 0.11 | nd | nd | nd |
| Destarched Corn | 0.03 | 0.05 | nd | nd | nd |
| Fiber | | | | | |

| | Sample Id | | | | | | | | |
|---|---|---|---|---|---|---|---|---|---|
| | C18:1 | C18 | C20 | C22 | C24 | C26 | C27 | C28 | C30 |
| | Alcohol | Alcohol | Alcohol | Alcohol | Alcohol | Alcohol | Alcohol | Alcohol | Alcohol |
| Corn | nd | nd | nd | nd | nd | nd | nd | nd | nd |
| Dewax | | | | | | | | | |
| Cake- | | | | | | | | | |
| liquid | | | | | | | | | |
| Corn | nd | nd | nd | nd | 0.02 | 0.01 | nd | nd | 0.01 |
| Dewax | | | | | | | | | |
| Cake- | | | | | | | | | |
| solid | | | | | | | | | |
| Rice | nd | nd | nd | nd | 0.01 | 0.02 | nd | 0.01 | 0.02 |
| FAD | | | | | | | | | |
| Cotton | nd | nd | 0.02 | 0.17 | 0.02 | 0.02 | nd | 0.06 | 0.03 |
| Seed | | | | | | | | | |
| DOD | | | | | | | | | |
| Soy | nd | nd | nd | nd | nd | nd | nd | 0.04 | 0.32 |
| Hulls | | | | | | | | | |
| Sunflower | nd | nd | 0.06 | 0.09 | 0.17 | 0.12 | nd | 0.08 | 0.11 |
| Hulls | | | | | | | | | |
| Linseed | nd | nd | 0.02 | 0.04 | 0.27 | 0.30 | 0.01 | 0.21 | 0.16 |
| DE | | | | | | | | | |
| Stearine | nd | nd | nd | nd | 0.02 | 0.01 | nd | 0.01 | 0.01 |
| 6117 | | | | | | | | | |
| Stearine | nd | nd | nd | 0.02 | 0.10 | 0.08 | nd | 0.06 | 0.03 |
| 6126 | | | | | | | | | |
| Stearine | nd | nd | nd | nd | 0.04 | 0.03 | nd | 0.03 | 0.02 |
| 6150 | | | | | | | | | |

TRANSESTERIFIED: Results are % w/w

| | Sample Id | | | | |
|---|---|---|---|---|---|
| | C24 Alcohol | C26 Alcohol | C27 Alcohol | C28 Alcohol | C30 Alcohol |
| Sun Oil Dewaxing | 1.09 | 0.97 | 0.03 | 0.53 | 0.33 |
| Cake C | | | | | |
| Sun Oil Dewaxing | 1.12 | 0.97 | 0.03 | 0.53 | 0.34 |
| Cake F | | | | | |
| Oelmuhle Leer Rape | 0.02 | 0.03 | 0.05 | 0.05 | 0.01 |
| Distillate | | | | | |
| Sun/Rape Distillate | 0.02 | 0.28 | 0.04 | 0.17 | 0.01 |
| Sunflower Distillate | 0.03 | 0.48 | nd | 0.04 | 0.03 |
| Canola A | 0.02 | 0.15 | 0.07 | 0.13 | 0.04 |
| Canola B | 0.02 | 0.15 | 0.09 | 0.13 | 0.03 |

### EXAMPLE 5

Analytical results obtained to date suggest streams derived during the processing of oilseeds to produce Vitamin E contain various levels of policosanols in various relative amounts of long chain fatty alcohols.

It should also be noted that the ratio of one policosanol to another varies depending on its source. Analysis of one ADM Vitamin E stream shows triacontanol to be the most abundant. In comparison, another ADM Vitamin E stream shows hexacosanol to be the most abundant.

A large portion of this research involved purification via column chromatography. Several sources have been investigated including various DODs and vitamin E streams. The results obtained thus far show the highest level of policosanols to be at least about 90%. In one investigation, policosanols were isolated from the Vitamin E stream using silica gel and a heptane:heptane/acetone elution. In one study, squalene was isolated in at least about 45% and the sterols isolated in at least about 60% purity. Another separation using Vitamin E stream and silica along with a very slow gradient elution of heptane:heptane/acetone provided at least about 62% squalene.

Solid-liquid extraction has also been an area of recent interest. The solvents of choice have been heptane, methanol, water, acetone, isopropanol and mixtures thereof. Quite interestingly, when two streams were subjected to the same extraction conditions (heptane:methanol:water at 94:5:1), the policosanols appear in the filtrate in one case and in the solid in the other. Studies performed with a third wax stream showed little or no separation in isopropanol but rather good separation in heptane:methanol:water.

Liquid-liquid extraction is another possible mode of purification. In an effort to better understand the solubility of policosanols, several tests were performed. Recent experiments have shown that policosanols are most soluble in ethers and aromatics.

Another possible starting source for policosanols is in the filtrates obtained from sterol isolation of DODs (deodorizer distillates). Several of the starting DODs showed amounts of policosanols which may have become enhanced with the isolation of sterols.

### EXAMPLE 6

A Soxhlet extraction process was performed using 1.5 L of methanol as a solvent for 20 h on 50 g of an ADM vitamin E stream. The obtained extract was left to cool to 25° C., and the solid was collected via Buchner filtration. The solids were then washed with methanol. The methanol extract solids were crystallized using toluene as solvent in a temperature range of 2 to 10° C. and dried in a vacuum oven at 25 inches Hg. The alcohols mixture (0.92 g) which was obtained had a purity amounting to 92.34%. The melting point ranges from 81.3 to 86.9° C. Table 9 shows the qualitative and quantitative composition of the higher molecular weight primary aliphatic alcohols provided by this procedure.

TABLE 9
Qualitative and Quantitative Composition

| Component | % of Each Alcohol |
|---|---|
| Total Policosanols | 92.34 |
| C22 OH | nd¹ |
| C23 OH | nd |
| C24 OH | 0.39 |
| C25 OH | 0.31 |
| C26 OH | 5.68 |
| C27 OH | 1.81 |
| C28 OH | 19.02 |
| C29 OH | 6.40 |
| C30 OH | 41.18 |
| C31 OH | 5.24 |
| C32 OH | 11.18 |
| C33 OH | 0.60 |
| C34 OH | 0.52 |

¹nd = not detected.

### EXAMPLE 7

A plant sample of vitamin E stream Cake Fraction from Mixed Tocopherol Dewaxing (33 g) was mixed with toluene (500 mL) and allowed to stir at 20° C. for 20 h. The mixture was subjected to Buchner filtration and rinsed with an additional 50 mL of toluene. The solids were dried in a vacuum oven at 25 inches Hg to provide 4.65 g of policosanols mixture with a purity of 77.64%. The composition of this mixture is outlined in Table 10.

TABLE 10
Qualitative and Quantitative Composition

| Component | % of Each Alcohol |
|---|---|
| Total Policosanols | 77.64 |
| C22 OH | 0.81 |
| C23 OH | 0.48 |
| C24 OH | 2.86 |
| C25 OH | 0.92 |
| C26 OH | 9.30 |
| C27 OH | 1.91 |
| C28 OH | 14.58 |
| C29 OH | 4.28 |
| C30 OH | 30.41 |
| C31 OH | 3.69 |
| C32 OH | 7.73 |
| C33 OH | 0.35 |
| C34 OH | 0.32 |

### EXAMPLE 8

A plant sample of vitamin E stream Cake Fraction from Mixed Tocopherol Dewaxing (16.7 g) was mixed with a 250 mL solution of 98:2 heptane:acetone (500 mL) and chilled to 5° C. for 18 h. The mixture was subjected to Buchner filtration and rinsed with an additional 30 mL of heptane:acetone. The solids were dried in a vacuum oven at 25 inches Hg to provide a policosanols mixture with a purity of 69.88%. The composition of this mixture is outlined in Table 11.

TABLE 11
Qualitative and Quantitative Composition

| Component | % of Each Alcohol |
|---|---|
| Total Policosanols | 69.88 |
| C22 OH | 6.33 |
| C23 OH | 2.84 |
| C24 OH | 9.82 |
| C25 OH | 1.95 |
| C26 OH | 12.00 |
| C27 OH | 1.76 |
| C28 OH | 10.21 |
| C29 OH | 2.64 |
| C30 OH | 18.00 |
| C31 OH | 2.04 |
| C32 OH | 3.93 |
| C33 OH | 0.18 |
| C34 OH | 0.17 |

### EXAMPLE 9

An analytical comparison of the isolated chromatographic samples and the commercially available materials (B&D, Asian) was performed. Table 12 shows the results obtained. Table 13 shows representative results obtained using complexation. An analytical comparison of the recrystallized policosanols according to the method of the present invention and commercially available samples (B&D, Asian) was also performed. Representative results are shown in Table 14.

TABLE 12
Comparison of Commercially Available and Chromatographically
Isolated Policosanols Samples.

| | | | | Policosanol |
|---|---|---|---|---|
| | | | Policosanol | Mix |
| | Standard | Standard | Mix Normal | Reversed |
| Component | B&D | Asian | Phase | Phase |
| Total Policosanols | 91.17 | 89.71 | 90.85 | 88.20 |
| C22 OH | nd¹ | nd | 0.66 | 7.24 |
| C23 OH | 0.01 | nd | 0.66 | 6.49 |
| C24 OH | 1.31 | 1.31 | 4.42 | 33.61 |
| C25 OH | 0.11 | 0.12 | 1.34 | 6.47 |
| C26 OH | 9.52 | 9.48 | 11.02 | 28.86 |
| C27 OH | 0.16 | 0.16 | 2.00 | 2.01 |
| C28 OH | 58.42 | 57.60 | 14.66 | 3.28 |
| C29 OH | 0.18 | 0.20 | 4.73 | 0.14 |
| C30 OH | 16.40 | 15.95 | 35.52 | 0.08 |
| C31 OH | 0.08 | 0.08 | 4.66 | nd |
| C32 OH | 3.84 | 3.76 | 10.28 | nd |
| C33 OH | 0.08 | 0.02 | 0.48 | nd |
| C34 OH | 1.06 | 1.03 | nd | nd |
| Total Tocopherols | 0.04 | 0.04 | 0.87 | 0.78 |
| Delta | 0.04 | 0.04 | nd | 0.17 |
| Beta | nd | nd | 0.14 | 0.13 |
| Gamma | nd | nd | 0.46 | 0.40 |
| Alpha | nd | nd | 0.27 | 0.09 |
| Total Sterols | 0.01 | nd | nd | nd |
| Brassicasterol | nd | nd | nd | nd |
| Campesterol | nd | nd | nd | nd |
| Campestanol | nd | nd | nd | nd |
| Stigmasterol | 0.01 | nd | nd | nd |
| Sitosterol | nd | nd | nd | nd |
| Sitostanol | nd | nd | nd | nd |
| Stanols | nd | nd | nd | nd |
| Total | 0.85 | 0.92 | nd | nd |
| Hydrocarbons | | | | |
| C22 | nd | nd | nd | nd |
| C24 | nd | 0.01 | nd | nd |
| C26 | 0.82 | 0.83 | nd | nd |
| C28 | nd | 0.05 | nd | nd |
| C30 | 0.02 | 0.02 | nd | nd |
| C32 | nd | nd | nd | nd |
| C34 | 0.01 | nd | nd | nd |
| C36 | nd | nd | nd | nd |

¹nd = not detected.

TABLE 13
Comparison of Complexation Extracts¹

| | | | Washing |
|---|---|---|---|
| | Starting | Washing with | with Methyl |
| Component | Complex Solid | Diisopropyl Ether | Ethyl Ketone |
| Total Policosanols | 39.28 | 76.02 | 64.41 |
| C22 OH | 0.62 | 1.44 | 2.49 |
| C23 OH | 0.48 | 1.24 | 1.87 |
| C24 OH | 2.73 | 8.21 | 11.01 |
| C25 OH | 0.79 | 2.46 | 3.00 |
| C26 OH | 6.37 | 18.99 | 19.68 |
| C27 OH | 1.11 | 2.84 | 2.37 |
| C28 OH | 7.23 | 14.90 | 9.96 |
| C29 OH | 1.98 | 3.25 | 1.97 |
| C30 OH | 12.91 | 17.05 | 9.06 |
| C31 OH | 1.53 | 1.80 | 0.98 |
| C32 OH | 3.25 | 3.50 | 1.82 |
| C33 OH | 0.15 | 0.16 | 0.09 |
| C34 OH | 0.14 | nd | 0.08 |
| Total Tocopherols | 0.08 | 0.04 | 5.25 |
| Delta | 0.02 | 0.04 | 1.36 |
| Beta | 0.06 | nd | 0.39 |
| Gamma | nd | nd | 2.68 |
| Alpha | nd | nd | 0.82 |
| Total Sterols | 0.05 | nd | 2.56 |
| Brassicasterol | nd | nd | nd |
| Campesterol | 0.03 | nd | 0.80 |
| Campestanol | nd | nd | nd |
| Stigmasterol | 0.02 | nd | 0.32 |
| Sitosterol | nd | nd | 1.44 |
| Sitostanol | nd | nd | nd |
| Stanols | nd | nd | nd |
| Total Hydrocarbons | 0.10 | 0.92 | 0.29 |
| C22 | nd | nd | nd |
| C24 | nd | 0.01 | nd |
| C26 | nd | 0.83 | nd |
| C28 | 0.03 | 0.05 | 0.13 |
| C30 | 0.07 | 0.02 | 0.16 |
| C32 | nd | nd | nd |
| C34 | nd | nd | nd |
| C36 | nd | nd | nd |

¹The solid was obtained from complexation of an MTD/heptane extract with CaBr₂ in MIBK. This solid was then washed with the listed solvent.

TABLE 14
Comparison of Commercially Available and
Recrystallized Policosanol Samples.

| | | | Isolated | |
|---|---|---|---|---|
| | Standard | Standard | Policosanol | Resubmission |
| Component | B&D | Asian | Mix | of Mix |
| Total | 91.17 | 89.71 | 90.24 | 92.34 |
| Policosanols | | | | |
| C22 OH | nd² | nd | nd | nd |
| C23 OH | 0.01 | nd | nd | nd |
| C24 OH | 1.31 | 1.31 | 0.39 | 0.39 |
| C25 OH | 0.11 | 0.12 | 0.34 | 0.31 |
| C26 OH | 9.52 | 9.48 | 5.87 | 5.68 |
| C27 OH | 0.16 | 0.16 | 1.76 | 1.81 |
| C28 OH | 58.42 | 57.60 | 18.71 | 19.02 |
| C29 OH | 0.18 | 0.20 | 6.30 | 6.40 |
| C30 OH | 16.40 | 15.95 | 39.92 | 41.18 |
| C31 OH | 0.08 | 0.08 | 5.04 | 5.24 |
| C32 OH | 3.84 | 3.76 | 10.87 | 11.18 |
| C33 OH | 0.08 | 0.02 | 0.54 | 0.60 |
| C34 OH | 1.06 | 1.03 | 0.49 | 0.52 |
| Total Tocopherols | 0.04 | 0.04 | 0.04 | 1.27 |
| Delta | 0.04 | 0.04 | 0.04 | 1.27 |
| Beta | nd | nd | nd | nd |
| Gamma | nd | nd | nd | nd |
| Alpha | nd | nd | nd | nd |
| Total Sterols | 0.01 | nd | 0.31 | 0.08 |
| Brassicasterol | nd | nd | nd | nd |
| Campesterol | nd | nd | nd | nd |
| Campestanol | nd | nd | nd | nd |
| Stigmasterol | 0.01 | nd | 0.12 | nd |
| Sitosterol | nd | nd | 0.19 | 0.08 |
| Sitostanol | nd | nd | 0.20 | 0.15 |
| Stanols | nd | nd | 0.20 | 0.15 |

²nd = not detected.

It will be appreciated by those skilled in the art that changes could be made to the embodiments described herein without departing from the broad concept of the invention. It is understood, therefore, that this invention is not limited to the particular embodiments disclosed, but is intended to cover modifications that are within the spirit and scope of the invention as defined by the claims.

We claim:

1. A method of isolating sterols comprising:
mixing a non-halogenated solvent with at least one sterol-containing material to form a mixture comprising dissolved sterols;
precipitating the dissolved sterols; and
recovering a precipitate of sterols.

2. The method of claim 1, wherein the mixture is cooled to precipitate the sterols.

3. The method of claim 2, wherein the mixture is heated prior to cooling.

4. The method of claim 1, wherein the precipitate comprises at least 0.2% policosanols by weight.

5. The method of claim 1, wherein the precipitate comprises at least 85% sterols by weight.

6. The method of claim 1, wherein the sterol-containing material comprises plant material.

7. The method of claim 6, wherein the sterol-containing material comprises at least one of crude tall oil soap and crude tall oil.

8. The method of claim 1, wherein the solvent and the sterol-containing material are present at a volumetric ratio ranging from 1:1 to a ratio of 20:1.

9. The method of claim 1, wherein the solvent comprises an organic solvent or a combination of organic solvents.

10. The method of claim 9, wherein the organic solvents are selected from the group consisting of heptane, hexane, cyclohexane, pentane, methanol, ethanol, isopropyl alcohol, acetone, methyl ethyl ketone, diethyl ether, ethyl acetate, acetone, and combinations thereof.

11. The method of claim 10, wherein the solvent further comprises water.

12. The method of claim 11, wherein the solvent comprises a combination of heptane, methanol, and water.

13. The method of claim 12, wherein the heptane is present at a volume ranging from 90% to 98% by volume.

14. The method of claim 12, wherein the methanol is present at a volume ranging from 1% to 5% by volume.

15. The method of claim 12, wherein the water is present at a volume ranging from 1% to 5% by volume.

16. The method of claim 1, further comprising the steps of:
mixing a recrystallization solvent with the precipitate to form a recrystallization mixture comprising dissolved sterols;
precipitating at least some of the dissolved sterols as a second precipitate; and
recovering the second precipitate, wherein the second precipitate consists of at least 95% sterols by weight.

17. The method of claim 16, wherein the recrystallization solvent is selected from the group consisting of heptane, hexane, cyclohexane, pentane, methanol, ethanol, isopropyl alcohol, acetone, methyl ethyl ketone, ether, ethyl acetate, and combinations thereof.

18. A composition formed by the process of claim 1.

19. The composition of claim 18, wherein the policosanols comprise at least 0.2% by weight of the composition.

20. A composition comprising policosanols and at least 98% sterols by weight.
